(19) United States
(12) Patent Application Publication
(10) Pub. No.: US 2007/0270947 A1
(43) Pub. Date: Nov. 22, 2007

(54) METHOD AND SYSTEM FOR MODIFYING AN INTRAOCULAR TELESCOPE

(75) Inventor: Gholam A. Peyman, Sun City, AZ (US)

Correspondence Address: BELL, BOYD, & LLOYD LLP, P.O. BOX 1135, CHICAGO, IL, 60690, US

(73) Assignee: MINU TELESYSTEMS LLC, Pittsboro, NC (US)

(21) Appl. No.: 11/750,177

(22) Filed: May 17, 2007

Related U.S. Application Data
(63) Continuation-in-part of application No. 11/384,998, filed on Mar. 20, 2006.
Application No. 11/384,998 is a continuation-in-part of application No. 11/151,978, filed on Jun. 14, 2005.
Application No. 11/384,998 is a continuation-in-part of application No. 10/455,788, filed on Jun. 6, 2003.
(63) Continuation-in-part of application No. 10/600,371, filed on Jun. 23, 2003, now Pat. No. 7,220,278.

(51) Int. Cl. A61F 2/16

(52) U.S. Cl. 623/6.34

(57) ABSTRACT

The present invention relates to a method and device for altering vision in an eye. The invention includes inserting into the eye a supplemental lens in series with an implanted Galilean telescopic intraocular lens. The Galilean telescopic intraocular lens is adapted to form a first image from a first field of vision and a portion of a second lens is adapted to form a second image from a second field of vision. Wherein the supplemental lens facilitates focusing light through the Galilean telescopic intraocular lens to form the first image and renders the second image substantially imperceptible.

### RELATED APPLICATIONS

This application is a continuation-in-part U.S. patent application Ser. No. 11/384,998, filed Mar. 20, 2006, entitled “INTRAOCULAR TELESCOPE,” which is continuation-in-part of U.S. application Ser. No. 11/151,978, filed Jun. 14, 2005, entitled “INTRAOCULAR TELESCOPE,” and a continuation-in-part of U.S. application Ser. No. 10/455,788, filed Jun. 6, 2003, entitled “TELEDIOPTIC LENS SYSTEM AND METHOD FOR USING THE SAME, and which is a continuation-in-part of U.S. application Ser. No. 10/600,371, filed Jun. 23, 2003, entitled “TELEDIOPTIC LENS SYSTEM AND METHOD FOR USING THE SAME,” now U.S. Pat. No. 7,220,278, which is a continuation-in-part of U.S. application Ser. No. 10/873,495, filed Jun. 23, 2004, and entitled “BIFOCAL INTRAOCULAR TELESCOPE FOR LOW VISION CORRECTION,” now abandoned, and which is a continuation-in-part of U.S. application Ser. No. 11/038,320, filed Jan. 17, 2005, entitled “BIFOCAL INTRAOCULAR TELESCOPE FOR LOW VISION CORRECTION,” now U.S. Pat. No. 7,186,266.” The entire contents of each of these applications are incorporated herein by reference.

### BACKGROUND

Macular degeneration has become one of the leading causes of blindness in adults. This disease affects the central retinal area known as the macula. The macula is responsible for acute vision. Macular degeneration can lead to a gradual or sudden loss of vision to the level of 20/200 or less. Commonly, loss of vision only affects the central macular area of about 0.25 to 4 square millimeters, and does not usually progress beyond this area, thereby leaving 95-99% of the retina unaffected. Thus, reading vision can be lost, while peripheral vision remains intact. This condition can be referred to as low vision.

Laser photocoagulation has been successful in treating macular degeneration certain instances. Telescopic systems that attach to eye glasses also have been used to improve vision in patients with macular degeneration.

U.S. Pat. Nos. 4,666,446 and 4,581,031, both to Koziol and Peyman, and both of which are incorporated by reference herein, each disclose intraocular lenses which are implanted in the eye in place of the natural lens to redirect the rays of light to minimize the adverse affect on vision caused by the macular degeneration of the eye. For example, U.S. Pat. No. 4,666,446 discloses an intraocular lens comprising a first portion including a diverging lens and a second portion including a converging lens.

U.S. Pat. No. 6,197,057 to Peyman and Koziol, the entire contents of which are herein incorporated by reference, relates to a lens system that combines a high plus lens with a plus and minus intraocular lens (IOL), so that the lens system works in a manner similar to a Galilean telescope. Generally the high plus lens is outside the eye (i.e., in glasses or spectacles or in a contact lens) and the plus and minus lens is an IOL that replaces or works in conjunction with the natural lens of the patient (See FIGS. 1 and 2).

U.S. Pat. Nos. 4,074,368 and 6,596,026, the entire contents of which are herein incorporated by reference, both disclose telescopic implants for implantation within an eye. These implants are designed to replace the natural lens in the eye with a telescope. They are rigid devices requiring a large incision in the eye to implant.

### SUMMARY

In one embodiment a method of altering vision in an eye is presented. The method includes the step of inserting into the eye a supplemental lens in series with an implanted Galilean telescopic intraocular lens. The Galilean telescopic intraocular lens adapted to form a first image from a first field of vision and a portion of a second lens adapted to form a second image from a second field of vision. Wherein the supplemental lens facilitates focusing light through the Galilean telescopic intraocular lens to form the first image and renders the second image substantially imperceptible.

In another embodiment, an implantable lens for converting a lens system from a multifocal lens system to a unifocal lens system is presented. The multifocal lens system includes a Galilean telescopic intraocular lens configured to form a first image from a first field of vision and a portion of a second lens configured to form a second image from a second field of vision. The implantable lens includes a plus lens adapted to be implanted in series with the Galilean telescopic intraocular lens such that the implantable lens facilitates focusing light through the Galilean telescopic intraocular lens to form a first image and renders the second image substantially imperceptible.

In another embodiment, a method of converting a lens system from a multifocal lens system to a unifocal lens system is presented. The multifocal lens system includes a Galilean telescopic intraocular lens configured to form a first image from a first field of vision and a portion of a second lens configured to form a second image from a second field of vision. The method includes the step of implanting into the eye a lens in series with the Galilean telescopic intraocular lens such that the lens facilitates focusing light through the Galilean telescopic intraocular lens to form the first image and renders the second image substantially imperceptible.

Additional features and advantages are described herein, and will be apparent from, the following Detailed Description and the figures.

### BRIEF DESCRIPTION OF THE FIGURES

FIG. 1 is a cross-sectional view in side elevation of a human eye with an intraocular implant according to a first embodiment of the present invention;

FIG. 2 is an enlarged cross-sectional view in side elevation of the telescope portion of the implant shown in FIG. 1 having a plus and a minus lens therein;

FIG. 3 is a top plan view of the intraocular implant shown in FIG. 1 prior to implantation;

FIG. 4 is a side elevational view of the intraocular implant shown in FIG. 3;

FIG. 5 is an enlarged cross-sectional view in side elevation of a modified telescope portion of the present invention using diffractive lenses;

FIG. 6 is a top plan view of an intraocular implant similar to that shown in FIGS. 3 and 4, but using U-shaped haptics;

FIG. 7 is a side elevational view of the intraocular implant shown in FIG. 6;

FIG. 8 is a cross-sectional view in side elevation of a human eye with an intraocular implant according to a second embodiment of the present invention with an artificial IOL substituted for the natural lens;

FIG. 9 is a cross-sectional view in side elevation of a human eye with an intraocular implant according to a third embodiment of the present invention used with the natural lens;

FIG. 10 is a cross-sectional view in side elevation of a human eye with an intraocular implant according to a fourth embodiment of the present invention;

FIG. 11 is a cross-sectional view in side elevation of a human eye with an intraocular implant according to a fifth embodiment of the present invention;

FIG. 12 is an enlarged cross-sectional view in side elevation of the telescope portion of the intraocular implant of FIG. 11 having a plus and a minus lens therein;

FIG. 13 is an enlarged cross-sectional view in side elevation of alternative telescope portion of the present invention for use with the embodiment of FIG. 11;

FIG. 14 is an enlarged cross-sectional view in side elevation of another alternative telescope portion for use with the embodiment of FIG. 11.

FIG. 15 is a cross-sectional view in side elevation of the embodiment of FIG. 1 further including a contact lens on the cornea;

FIG. 16 is a cross-sectional view in side elevation of the embodiment of FIG. 1 further including an external spectacle;

FIG. 17 is a top plan view of a bifocal contact lens;

FIG. 18 is a perspective view of an alternative telescope portion for providing a teledioptic lens system;

FIG. 19 is an elevational side view in section of an external spectacle with an opaque portion or member blocking light from passing through the central portion of the spectacle;

FIG. 20 is an elevational side view in section of the spectacles of FIG. 19 with the opaque portion moved away from the central portion of the spectacle;

FIG. 21 is an elevational side view of a telescopic lens system according to another embodiment of the present invention;

FIG. 22 is a front view of a telescopic lens system according to another embodiment of the present invention;

FIG. 23 is an elevational side view in section of the lens system of FIG. 22 taken along lines 23-23;

FIG. 24 is a side view in section of an eye with the natural lens removed and the lens system of FIG. 23 implanted therein;

FIG. 25 is a front view of a telescopic lens system according to another embodiment of the present invention;

FIG. 26 is a side view of the telescopic lens system of FIG. 25;

FIG. 27 is a side view in section of the telescopic lens system of FIG. 26 inserted in the eye and being used in conjunction with external spectacles;

FIG. 28 illustrates a supplemental lens that is configured to be positioned in the eye such that it facilitates focusing light through a Galilean telescopic intraocular lens to form a first image and renders a second image formed by a second lens substantially imperceptible;

FIG. 29 is another embodiment of the supplemental lens of FIG. 28 coupled to the Galilean telescopic; and

FIG. 30 another embodiment of the supplemental lens of FIG. 28 coupled to the iris.

### DETAILED DESCRIPTION

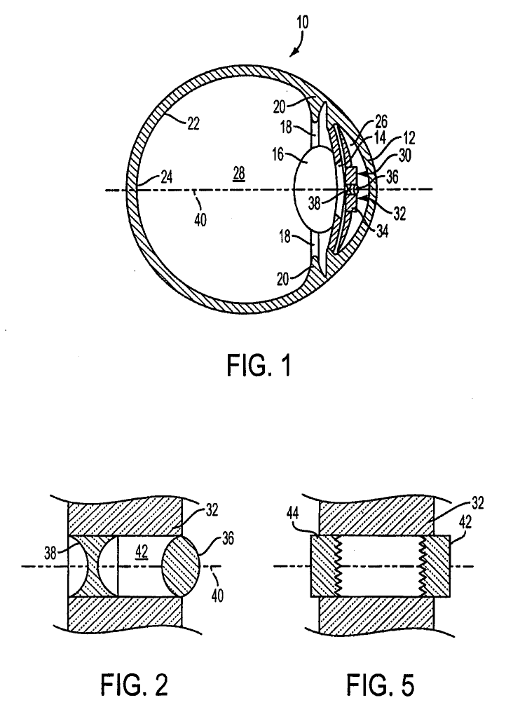

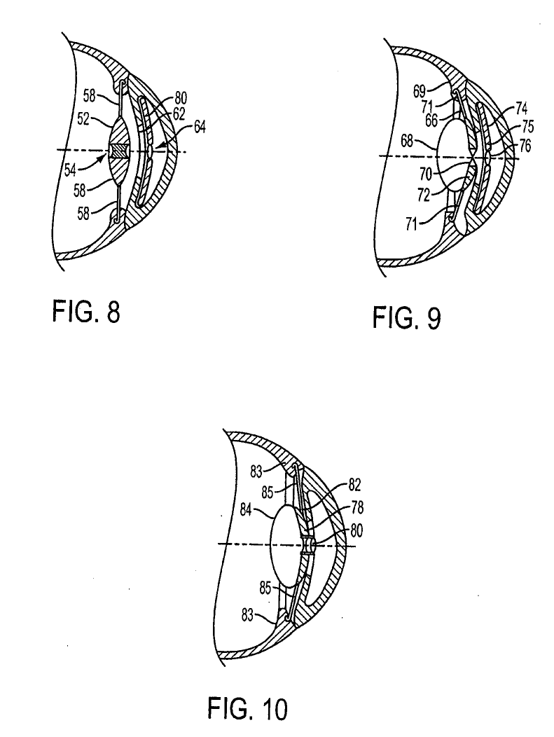

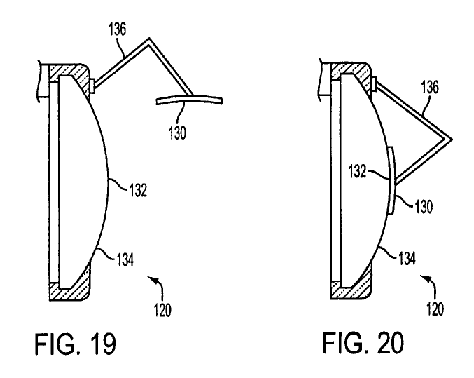

FIGS. 1-28 illustrate a Galilean telescopic intraocular lens or an intraocular telescopic lens implant 30 can be implanted in the eye 10. The intraocular telescopic lens implant 30 can have a telescope portion 32 surrounded by a substantially transparent peripheral portion 34.

The telescope portion 32 allows light to pass therethrough and has a bi-convex converging, or plus lens 36 and a bi-concave diverging, or minus lens 38. The lenses 36, 38 are aligned along an optical axis 40 to form a Galilean telescope. Preferably, the lenses are about 1-2 mm in diameter, but can be any diameter. The diverging lens 38 can have a refractive index between −30 and −90 diopters, as measured in water or any other suitable index. The converging lens 36 can have a refractive index between +30 and +80 diopters, as measured in water or any other suitable index. Please note that the telescope portion can have any suitable configuration and/or refractive properties desirable and is not limited to any specific description herein.

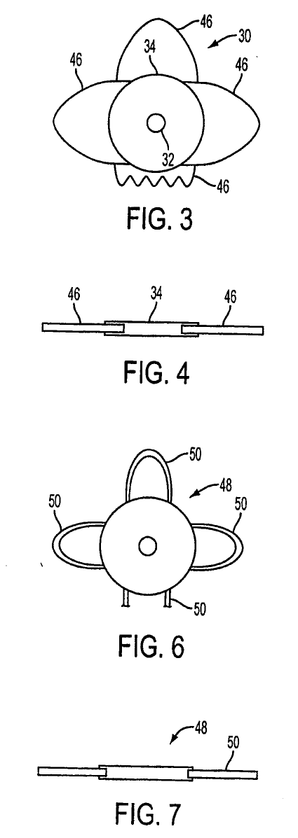

FIGS. 3 and 4 illustrate the intraocular telescopic implant 30 prior to implantation. The substantially circular peripheral portion 34 surrounding or substantially surrounding the telescope portion 32 is made of a biocompatible, transparent, optical material. Peripheral portion 34 is preferably flexible, but can be rigid or partially rigid and partially flexible or any other suitable configuration. The peripheral portion can have a diameter of approximately 2 to 6.5 mm, and a thickness of approximately 0.05 to 1 mm, but can have any suitable dimensions. The peripheral portion can supplement the natural lens or can replace the natural lens. Therefore, the peripheral portion of the lens can have no refractive power (i.e., allow light to pass therethrough substantially without altering the path thereof) or can have any suitable refractive power to correct for refractive error or properly focus the light onto the retina if the natural lens is partially, substantially or completely removed.

The peripheral portion 34 may also have varying thickness and refractive power to correct for any astigmatism in the eye. Further, the peripheral portion 34 can have multiple focal adjustments—i.e., bifocal—to correct for and provide multiple refractive corrections.

Generally, to implant the intraocular telescopic implant in the eye, an incision can be made in the eye through the use of a microkeratome, laser, or other suitable surgical device. The implant 30 can be folded or rolled up, and inserted into the eye through the incision. The implant 30 is allowed to unfold or unroll (if folded), and haptics 46 can affix the implant into interior of the eye 10. Since the implant 30 can be foldable, the incision can be relatively small. This is beneficial because any incision in the eye can cause astigmatisms in the eye and may require varying healing periods. The implant 30 can be implanted into the anterior and/or posterior chamber, as desired or implanted into the capsular bag.

Generally, light rays enter the eye from the central field of vision substantially parallel to the axis 40 of the telescopic implant 30. Because they are parallel to the axis of the telescope, the rays enter the telescope and are magnified and projected onto the retina to provide enhanced acute vision for the central field of vision. At the same time, light rays from the peripheral field are unobstructed by the transparent peripheral portion 34 of the lens implant so that the patient retains unrestricted peripheral vision. Furthermore, because the peripheral portion of the implant is generally transparent, a doctor examining a patient's retina has an unobstructed view of the retina.

The lenses 36, 38 illustrated in FIGS. 1-2 are conventional bi-convex and bi-concave lenses. The conventional lenses are refractive lenses—i.e. they utilize refraction to modify how light propagates through the lenses to change the focal point of the lenses. The lenses in the telescopic implant 30, however, may have any desirable shape or configuration.

Furthermore, the lenses in telescope portion 32 can use diffractive technology, if desired. Diffractive lenses, such as Fresnel lenses, utilize diffraction to modify how light propagates through the lenses to change the focal point of the lenses. Diffractive lenses are advantageous because they are very thin as compared to conventional refractive lenses. Other suitable lenses include those made by ThinOptx, Inc. of Abingdon, Va. ThinOptx, Inc. manufactures intraocular lenses that are approximately 100 microns thick with +/−25 diopters of correction. Further details regarding these lenses are found in U.S. Pat. Nos. 6,666,887 and 6,096,077, which are hereby incorporated by reference in their entirety. When using technology such as this, the telescope portion can be about 2-3 mm, preferably about 2 mm thick, but can have any suitable dimensions.

As shown in FIG. 18, lens and lens in the telescopic portion can be connected using one or multiple struts, for example struts 130, 132, 134, 136. Each strut can be attached to the periphery 138 of lens 38 (in any manner, such as adhesive or any other suitable means) and extends to the periphery 140 of lens 36 and attaches thereto in the same or substantially similar manner; however, the struts do not necessarily need to be connected at the periphery and can be attaches in any manner desired. Furthermore, the telescope portion 129 can have any suitable number of struts. For example, the telescope portion can have as few as one strut or as many as desirable, or the struts can be continuous, forming a cylinder or a cylinder with spaces or holes.

The struts are preferably formed from a material that can be flexible, such as the material disclosed above or portion 34 or any other suitable material. By forming the telescope portion 129 in this manner, natural fluid from the eye can flow between the lenses of the telescope portion. Additionally, the entire structure including the telescope portion 129 and peripheral portion 82 can be folded when inserted into the eye and unfolded after entry into the appropriate chamber. This flexibility allows the implant 78 to be inserted into a smaller incision in the surface of the eye, thus reducing possible damage to the eye.

The struts that allow natural fluid to flow therebetween. That is, fluid from the eye can pass between the lenses and to other portions of the internal portion of the eye. However, the lenses can be positioned in any manner that would allow fluid to pass therethrough. Furthermore, although generally preferable, it is not necessary to have fluid pass between the lenses and the lenses can be coupled (on not) in any suitable manner, including separated by any desired substances or a vacuum. Preferably, as described above, the struts are flexible, so that the entire lens system, including the telescope portion can be inserted into the smallest possible incision; however, the struts can be any suitable configuration (including rigid, if desired) and the telescope portion can have any number of struts desired.

When implanted, the telescope portion can extend through the iris; however, it is noted that the telescope portion does not necessarily need to extend through the iris and it can be situated in the eye in any suitable manner

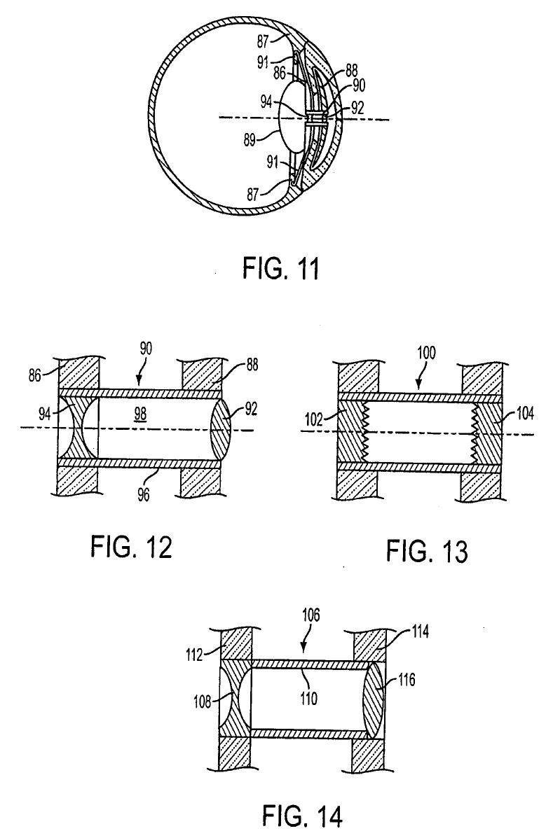

Although preferable, it is not necessary for the telescope portion 80 described in FIGS. 12-14 and telescope portion 129 described in FIG. 18 to be used with peripheral portions. For example, the telescope portion can be used with one peripheral portion, as disclosed in FIG. 10, two peripheral portions as disclosed in FIG. 11 or no peripheral portions. When used with no peripheral portions, the telescopic portion can be affixed inside the eye in any suitable manner, such as with haptics, adhesive or friction. Additionally, the telescopic portion can be affixed to the natural lens, an artificial lens or any other suitable structure (natural or artificial) inside the eye.

The implantation of the lenses described herein does not necessarily need to occur during one operating procedure and can occur over a predetermined period of time (e.g., seconds, minutes, days, weeks, months or years)

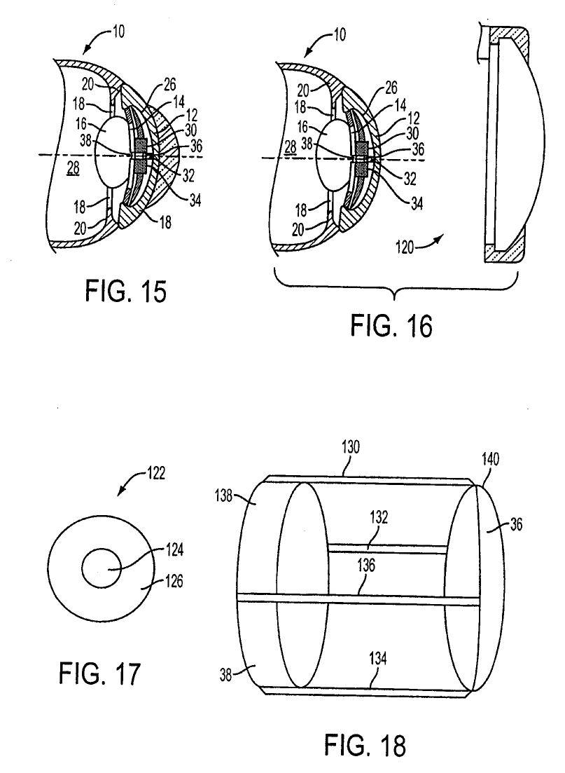

Generally, the telescopic IOL is used in conjunction with a supplemental lens located outside the eye. FIGS. 15 and 16 illustrate this. In FIG. 15, a supplemental plus contact lens 118 is placed on the cornea 12. In FIG. 16, a supplemental spectacle with two plus lenses 120 is placed in the visual path. In both cases, the lenses 118, 120 have a positive refractive index. The use of supplemental lenses outside the eye allows for smaller implants inside the eye. Further, the use of supplemental lenses allows the construction and operation of the implants to be tailored to particular patients' desires. For instance, many individuals have a preferable reading distance (typically between 20 and 50 cm away from the eye) and a supplemental lens allows the focal distance to be tailored to coincide with an individual's preferred reading distance.

The peripheral portion 126 (of either the contact lens or the spectacles) can provide refractive correction for far vision; however, the peripheral portion 126 can have any refractive properties desired. For example, the peripheral portion can be used to correct myopia, hyperopia, astigmatism, presybyopia, or any other vision error, or the peripheral portion of the lens can have no refractive properties, thus allowing a patient with acceptable peripheral vision to see with no correction (other than the telescopic central correction).

Additionally, a supplemental lens can be inserted into a portion of the eye. For example, a supplemental lens can be inserted partially into the cornea, between layers of the cornea, in the anterior chamber of the cornea and/or in the poster chamber of the cornea.

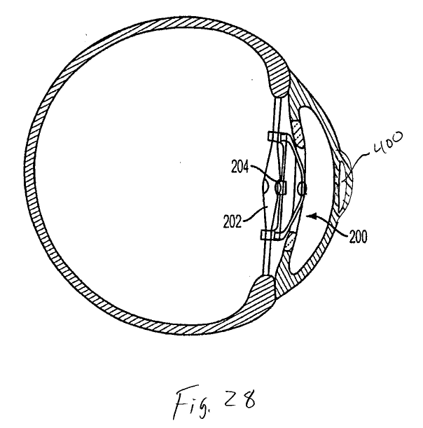

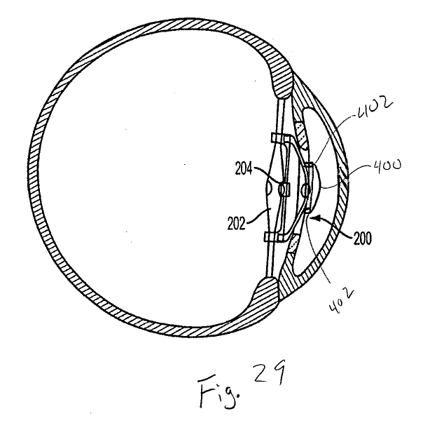

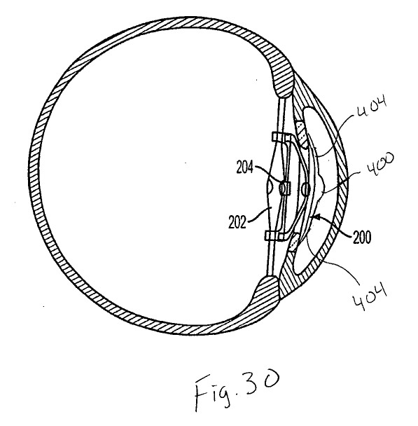

FIGS. 28-30 illustrate a supplemental lens 400 that is configured or adapted to be positioned relative to a Galilean telescopic intraocular lens 200 that has been implanted in the eye. Lens 400 is generally a plus lens that focuses light into and through the Galilean telescopic intraocular lens 200 to form a first image. However, the supplemental lens can be any shape or configuration desired.

Preferably, the supplemental lens is similar to the supplemental lens 200 described herein; however, the supplemental lens can be any suitable lens and does not necessarily need to be a plus lens. The supplemental lens works in conjunction with the telescopic portion as described herein.

Furthermore, the supplemental lens can be attached to unattached to the telescopic portion as shown in FIG. 29. For example, additional struts 402 or other coupling members can be used to attach the supplemental lens to any portion or lens or multiple portions or lenses in the telescopic portion 200. Additionally, the supplemental lens can have haptics 404 or other means to couple or attach to any portion of the eye. For example, the supplemental lens can be attached to the iris or the zonules or any other suitable portion of the eye.

Preferably, the supplemental lens 400 is configured and positioned in series with the telescopic portion 200, thus magnifying an image on the retina. The supplemental lens can be positioned such that the magnification of the image in permanent or semi-permanent. In other words, the patient will only be able to see the magnified image and the peripheral portion (if in place) of the lens system will be rendered useless or substantially useless. Such a system would allow someone not interested in far vision to always be able to see close objects without the need for supplemental lenses or without having multifocal properties in their lens systems.

The insertion of the supplemental lens could be reversible, such that the lens supplemental lens could be removed at a given time to allow the patient to see far without the use of the telescopic portion. The patient could then use an exterior supplemental lens to see close or near objects, as described herein or, if the lens system is multifocal, to use it as such.

Lens 400 can work in conjunction with each of the herein described embodiments.

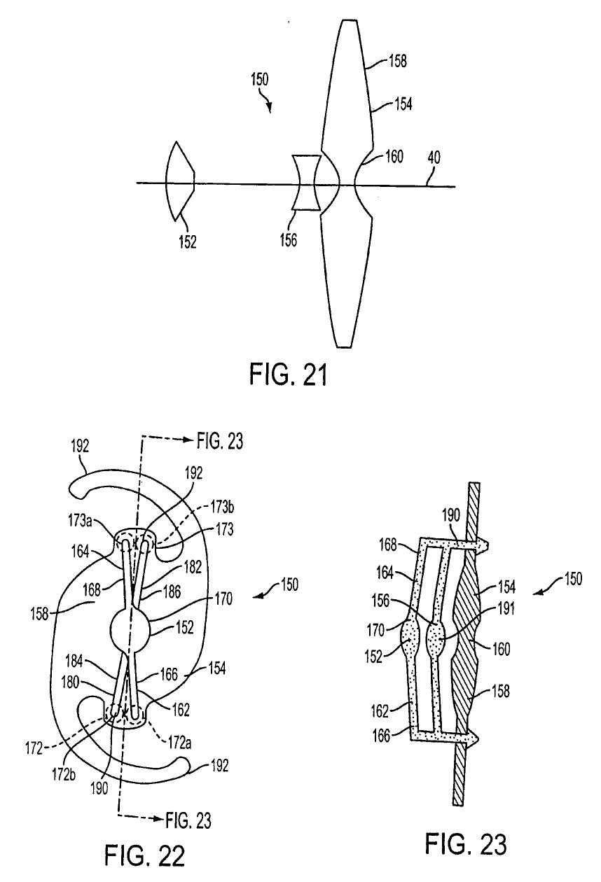

### Embodiments of FIGS. 21-24

FIGS. 21-24 illustrate additional embodiments of the present invention, wherein the telescopic intraocular lens system 150 includes at least three lenses, a first lens 152, a second lens 154 and a third lens 156. As with the above described systems, the present lens system preferable includes each of the lenses positioned substantially in series with each of the other lenses along the main optical axis of the eye.

Preferably first lens 152 is a plus lens (i.e., a biconvex asphere) and is positioned, relative the second and third lenses, closest to the cornea or the front of the eye. The first lens is preferable formed from PMMA; but can be formed from any suitable material(s). First lens 152 can also have any configuration desired and/or change or correct the refractive properties of the eye in any manner desired, that is, first lens 152 can be biconvex, biconcave, toric or any suitable combination thereof. First lens 152 preferably has a diameter between about 1.0 mm and about 1.5 mm, but can have any suitable diameter.

Second lens 154 is preferably a multifocal or bifocal lens. That is the second lens preferably has two different zones for focusing light; however, it is noted that the second lens can have any number of zones of portions capable of focusing, including one or more than two. Second lens 154 is preferably positioned, relative to the first and third lenses closest to the natural lens of the eye, if present or closest to the rear of the eye. Peripheral portion 158 of the lens 154 is a generally a converging lens (i.e., a biconvex asphere). Peripheral portion 158 preferably has a diameter about 6.0 mm; but can have any suitable diameter. The central portion 160, is a diverging lens with a high negative refractive index i.e. a biconcave lens) and has a diameter of about 1.0 mm, but can have any suitable diameter. However, it is noted that the both the central portion and the peripheral portion can be any suitable configuration desired and/or be adapted to change or correct the refractive properties of the eye in any manner desired or have no corrective properties, thus allowing light to merely pass therethrough. Second lens is preferably formed from PHMA (HEMA), but can be formed from any suitable material(s). Additionally, second lens 154 is preferably positioned in series or substantially in series with lens 152 and substantially along the main optical axis of the eye.

As shown in FIG. 21, third lens 156 is preferably a minus lens (i.e., biconcave) and is preferably positioned substantially between the first and second lenses, along the main optical axis. As with the first and second lenses, third lens can be any suitable configuration desired and/or be adapted to change or correct the refractive properties of the eye in any manner desired. Additionally, as with the first lens, third lens 156 is preferably formed from PMMA, but can be formed from any other suitable material(s). Third lens 156 preferably has a diameter between about 1.0 mm and about 1.5 mm, but can have any suitable diameter.

As shown in FIGS. 22-24, first lens 152 can be coupled to second lens 154 using two struts or coupling members 162 and 164. Preferably, struts 162 and 164 have a first portion 166 and 168, respectively, that each extends radially outwardly from the periphery 170 of lens 152. At about the periphery of the second lens 156 two protrusions or extensions 172 and 173 extend. The protrusions are about 180° offset from each other. Protrusion 172 has two openings 172a and 172b and protrusion 173 has two openings 173a and 173 that each extend through a respective protrusion. Second portions 174 and 176 of struts 162 and 164, respectively, extend substantially perpendicularly or at angle slightly greater than 90° to the first portion of each strut (substantially parallel to the main optical axis) and toward a respective protrusion on the second lens, coupling to the second lens at a substantially perpendicular angle. Each strut extends through a respective opening in the protrusions, allowing the struts to couple thereto. It is noted that the struts can couple the first lens to the second lens in any manner desired and do not necessarily need to be configured as described herein and/or do not need to couple to the lens as described herein.

Additionally, the first lens does not necessarily need to couple to the second lens and can couple to the third lens if desired. Furthermore, it is not necessary for the first lens to couple to the second lens using two struts and the first lens can couple to the second (and/or third) lens using as many or as few (one) struts as desired.

Third lens 156 preferably couples to second lens 154 using two struts 180 and 182. Structurally, struts 180 and 182 are substantially similar to struts 162 and 164. That is, struts 180 and 182 preferably each have a first portion 184 and 186, respectively, and a second portion 188 and 190, respectively, Each first portion extends radially outwardly from the periphery 191 of the third lens and each second portion 188 and 190 extend from a respective first portion substantially at a 90° degree angle or substantially parallel to the main optical axis and couples to the second lens through a opening or hole therein. As shown in FIG. 22, struts 180 and 182 extend from the third lens periphery slightly radially offset from struts 162 and 164. Thus, struts 180 and 182 can couple to the second lens at a different peripheral portion than struts 162 and 164.

As with struts 162 and 164 the struts can couple the third lens to the second lens in any manner desired and do not necessarily need to be configured as described herein and/or do not need to couple to the lens as described herein. Additionally, the third lens does not necessarily need to couple to the second lens and can couple to the first lens if desired. Furthermore, it is not necessary for the third lens to couple to the second lens using two struts and the third lens can couple to the second (and/or first) lens using as many or as few (one) struts as desired.

Extending from the periphery of second lens 154 are haptics 192. Although two J-shaped haptics are shown, the present device can have nay number of haptics and the haptics 192 can be any suitable configuration desired. Additionally, any or all of lenses 152, 154 and 156 can have any number of haptics extending thereof, or can be positioned and/or coupled inside of the eye in any manner desired.

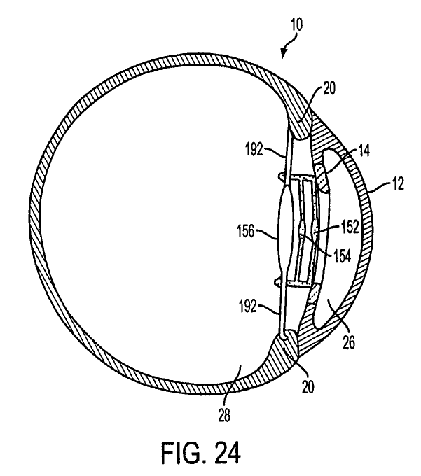

As shown in FIG. 24, intraocular lens system 150 is positioned in the posterior chamber of the eye and replaces the natural lens of the eye. Preferably haptics 192 couple the lens system to the eye by piercing the ciliary sulcus 20 of the eye. However, as stated above the lens system can be positioned in the eye in any manner desired. Each of the lenses 152, 154 and 156 is preferably positioned substantially centered around the main optical axis of the eye in series with each other lens, forming a telescopic or teledioptic lens system.

This system type of system allows light traveling through the peripheral portion of the eye to be focused on the retina by the peripheral area of the of the second lens and/or the natural and/or an artificial lens and light traveling through the central portion of the cornea to be magnified by the series of lenses and/or the natural and/or an artificial lens, thus forming a bifocal or multifocal lens system. More specifically, this type of lens system allows the patient to view far objects and near objects without the aid of external lenses. However, it is noted that this type of lens system is suitable for use with external lenses (e.g., glasses or contacts), if desired.

Additionally it is noted that the lens system described herein can be used to supplement or to replace the natural lens of the eye. Additionally, the system described herein is not limited to be positioned as shown herein, that is, all lenses positioned in the posterior chamber. Each lens can be positioned in either the anterior or posterior chamber of the eye, or positioned in the pupil spanning both the anterior and the posterior chambers. For example, (1) first lens 152 can be positioned in the anterior chamber and second lens 154 and third lens 156 can be positioned in the posterior chamber; (2) the first and third lenses can be positioned in the anterior chamber and the second lens can be positioned in the posterior chamber; or (3) the first, second and third lenses can each be positioned in the anterior chamber.

In examples (1) and (2) of the above paragraph, it may be beneficial to couple the first lens directly to the third lens and/or the third lens directly to the second lens. Furthermore, the coupling member or struts in such a case can be configured such that they can pass though the pupil and not the iris, see for example, FIG. 18. However, it is noted that the lenses can couple to each other in any manner desired (including passing through the iris) and also that if desired the lenses do not need to be coupled together but can merely be positioned within the eye at the appropriate position relative to each other lens.

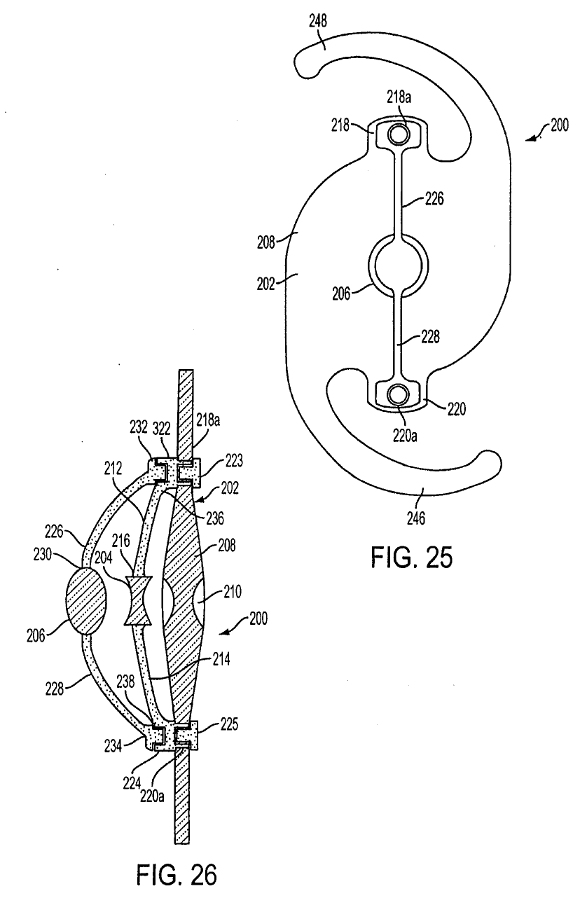

### Embodiment of FIGS. 25-27

FIGS. 25-27 illustrate another embodiment type of telescopic IOL, wherein a lens system 200 includes a first lens 202, a second lens 204 and a third lens 206; however, it is noted that this system is not limited to a specific number of lenses and it can have any suitable number of plus, minus, toric or other lenses. For example, lenses 204 and 206 can each be eliminated or divided into additional plus and/or minus lenses to create the desired refractive properties. Each lens preferably has a refractive index of about 1.48, but can have any suitable refractive index.

First lens 202 is similar to lens 154 in that lens 202 is a multifocal or bifocal lens and can replace the natural and/or existing artificial lens or it can be used in conjunction with the natural or artificial lens(es) (i.e., a piggyback lens). That is, lens 202 can piggyback onto existing intraocular lenses already in a patient's eye (for example, a polymer lens), the natural lens or any new lens positioned in the eye. When used as a piggyback lens, lens 202 can be positioned on the posterior surface, anterior surface or any other portion of the existing natural or artificial lens desired. Using lens 202 as a piggyback lens will allow the existing lens to be completely emetrope or substantially completely emetrope.

Lens 202 preferably has a peripheral portion 208 and a central portion 210, such that lens 202 has two different zones for focusing light; however, it is noted that lens 202 can have any number of zones of portions capable of focusing, including one or more than two. Lens 202 is preferably positioned, relative to lenses 204 and 206, closest to the natural lens of the eye, if present or closest to the rear of the eye if the natural lens has been removed. However, lenses 204, 206 and 202 can be positioned in any order and in any suitable portion of the eye. Peripheral portion 208 of lens 202 is a generally a converging lens (i.e., a biconvex asphere). Peripheral portion 208 preferably has a diameter about 6.0 mm; but can have any suitable diameter.

The central portion 210, is a preferably a diverging lens with a high negative refractive index (i.e. a biconcave lens or any combination of lenses having a power of about −790 diopters) and has a diameter of about 1.0 mm to about 1.5 mm, but can have any suitable diameter and/or power. Additionally, it is noted that both the central portion and the peripheral portion can be any suitable configuration desired and/or be adapted to change or correct the refractive properties of the eye in any manner desired or have no corrective properties, thus allowing light to merely pass therethrough. Lens 202 is preferably formed from PHMA (HEMA), but can be formed from any suitable material(s). Preferably, lens 202 is formed from material that is flexible so that it can be folded for insertion into the eye; however foldability is not necessary. Additionally, lens 202 is preferably positioned in series or substantially in series with lenses 204 and 206 and substantially along the main optical axis of the eye. If desired each of the herein described lenses can be formed of any suitable flexible material to allow them to be bent or folded to facilitate insertion into the eye.

Lens 204 is preferably a diverging lens with a high negative refractive index (i.e. a biconcave lens or any combination of lenses having a power of about −790 diopters) and has a diameter of about 1.0 mm to about 1.5 mm, but can have any suitable diameter and/or power. However, it is noted that lens 204 can be any suitable configuration desired and/or be adapted to change or correct the refractive properties of the eye in any manner desired or have no corrective properties, thus allowing light to merely pass therethrough. Lens 204 is preferably formed from PHMA (HEMA), but can be formed from any suitable material(s).

Lens 206 is preferably a preferably a converging lens (i.e., a biconvex asphere or any combination of lenses having a power of about 250 diopters) and has a diameter of about 1.0 mm to about 1.5 mm, but can have any suitable diameter and/or power. However, it is noted that lens 206 can be any suitable configuration desired and/or be adapted to change or correct the refractive properties of the eye in any manner desired or have no corrective properties, thus allowing light to merely pass therethrough. Lens 204 is preferably formed from PHMA (HEMA), but can be formed from any suitable material(s).

As with the embodiments described above, this lens system 200 is configured to form or project multiple images in the eye. For example, portion 208 of lens 202 is configured to focus light from the peripheral field of vision on the retina to form a first image in the eye, and lenses 204 and 206 and portion 210 are configured to focus light from the central visual field on the retina to form a second image in the eye.

Preferably, portion 208 has about the same refractive properties as a normal or natural lens and operates by itself or in conjunction with the natural lens to focus the peripheral light passing therethrough onto the retina to allow a patient to view far objects; however, it is noted that portion 208 can operate in any suitable manner, including focusing on close objects.

While focusing on far objects, preferably the image produced by lenses 204 and 206 and portion 210 is diverged and does not produce a suitable image on the retina. However, it is noted that if desired, the image produced by lens 204 and lens 206 and portion 210 and the image or images produced by lens 208 can by projected onto the retina at substantially the same time to form a substantially continuous image or any other suitable combination of images.

Similarly, when focusing on near objects through lenses 204 and 206 and portion 210, the imaged produced by lens 208 is converged such that the image is not suitably projected onto the retina, and the image produced by lens 204 and 206 and portion 210 is an enlarged or magnified image that is projected onto the retina. However, as described above, any suitable combination of images can be produced.

Lens 204 is preferably connected or coupled to lens 202 using struts 212 and 214. Preferably, struts 212 and 214 each extends radially outwardly from the periphery 216 of lens 204 and curve toward lens 202. At about the periphery of the second lens 202 two protrusions or extensions 218 and 220 extend. The protrusions are about 180° offset from each other. Protrusion 218 has an opening 218a, protrusion 220 has an opening 220a that each extend through a respective protrusion. Struts 212 and 214 have a respective portion 222 and 224 that extend at any suitable angle from the first portion of each strut (preferably substantially parallel to the main optical axis) and toward a respective protrusion on lens 202, coupling lens 204 to lens 202. Each strut extends through a respective opening in the protrusions, allowing the struts to couple thereto. Plugs or connectors 223 and 225 are inserted into the opposite side of opening 218a and 220a, respectively and facilitate the coupling of struts 212 and 214 to lens 202. It is noted that the struts can couple the first lens to the second lens in any manner desired. For example, the strut can be adhered, bonded, frictional fit or coupled in any other suitable manner. Additionally, the struts do not necessarily need to be configured as described.

Lens 206 is preferably connected or coupled to lens 204 using struts 226 and 228. Preferably, struts 226 and 228 each extends radially outwardly from the periphery 230 of lens 206. Struts 212 and 214 each have a respective opening 232 and 234 that are configured to receive a protrusion or portion 236 and 238 or struts 226 and 228, respectively. Portions 236 and 238 extend substantially parallel to the main optical axis and couple lens 206 to lens 204. It is noted that the struts can couple lens 206 to lens 208 in any manner desired. For example, the strut can be adhered, bonded, frictional fit or coupled in any other suitable manner. Additionally, the struts do not necessarily need to be configured as described. For example, each lens can couple using any number of struts desired or by any other connection desired and the struts themselves can have any suitable configuration.

Additionally, it is noted that lenses 202, 204 and 206 do not each need to be coupled together as described herein and any one of each lens can be coupled to any other lens or coupled to no lenses.

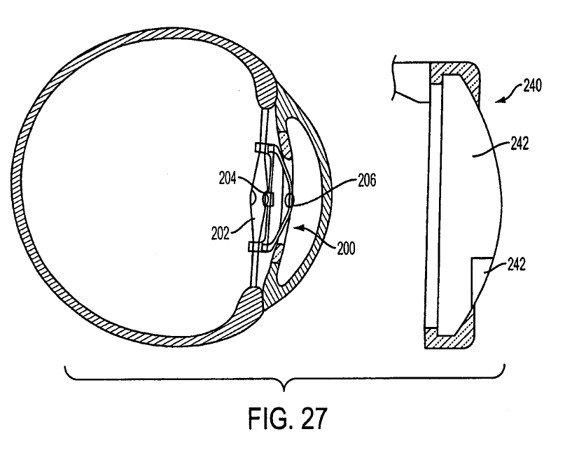

As shown in FIG. 27, external lens 240 is preferably used in conjunction with lens system 200, although the lens system described above can operate without the use of an external spectacle or lens. External lens 240 can be any suitable external lens, such as spectacles, glasses, contact lenses or any other suitable lens that is easily positioned in front of or proximal to the eye. Preferably lens 240 is a converging lens with a power of about 20 diopters, but lens can have any suitable power or no power. Additionally, lens 240 can be partially or fully implanted into the cornea or other portion of the eye is desired. For example, intrastromal corneal inlays and subepithelial corneal onlays are both suitable for use in conjunction with lenses 202, 204 and 206.

Lens 240 is preferably a plus lens or converging lens that is configured to facilitate focusing of light through lens 204 and lens 206 and portion 210. In other words, when lens 240 is positioned adjacent or in front of the eye, light is focused through lens 204 and lens 206 and portion 210, thus forming an image on the retina that is magnified about 3.5×; however, it is noted that any suitable combination of lenses can be used to great any magnification desired. When lens 240 is removed, light passes through the cornea and is focused on the retina by peripheral portion 208, as described above.

In another embodiment, lens 204 and lens 206 and portion 210 can be polarized to permit light oriented in a first direction to pass therethrough and portion 208 can be polarized to permit light oriented in a second to pass therethrough. Preferably, the polarized first direction is 90° offset from the polarized second direction.

Additionally, lens 240 can have a first portion 242 and a second portion 244 that are similarly polarized. That is, for example, second portion 244 can be polarized to permit light having substantially the same orientation as lens 204 and lens 206 and portion 210 and first portion 242 can be polarized to permit light having substantially the same orientation as portion 208. Therefore, when the patient views an object through the first portion 242 the object is projected onto the retina through portion 208 and substantially no light passes through lens 204 and lens 206 and portion 210. Conversely, when an object is viewed through second portion 244 the object is projected onto the retina through lens 204 and lens 206 and portion 210 and substantially no light passes through portion 208. It is noted that is not necessary for lens 240 to merely have two portions. Lens 240 can have any number of portions desired, including one and more than two. For example, if lens 240 has only one portion, lens 204 and lens 206 and portion 210 can be polarized to substantially match the polarization of lens 240. In this instance, when the lens is adjacent the eye the light would be polarized and pass through lens 204 and lens 206 and portion 210, and when the lens 240 is removed or not adjacent the eye the light would pass through portion 208. Portion 208 can be the polarized portion (i.e., 204 and lens 206 and portion 210 would not be polarized) in this example, if desired.

Furthermore, lens 202 preferably has haptics 246 and 248 extending therefrom to couple the lens system 200 in the eye. Haptics 246 and 248 are merely exemplary and lens system 200 can be positioned in the eye in any suitable manner.

Preferably, lens 204 and 206 are coupled together prior to insertion into the eye and/or prior to examination a patient. By fixing these lenses together, it is possible to determine the optimal distance to produce a telescopic effect. Additionally, lens 202 can be coupled substantially at the same time to accurately fix the optimal distance. However, it is noted that the lenses 202, 204 and 206 can be coupled together at anytime before or after examination of the patient.

It is noted that any of the lenses described herein can have any desired configuration and/or can be configured to correct for any desired optical aberration.

### EXAMPLES

The following tables show specific examples for the dimensions and design of an intraocular lens system according to the present invention. These examples were evaluated on an axis and a small field angle in 555 nm light and conditions within the eye (35° C. and surrounded by media with index of refraction of 1.336). The in situ power of the peripheral part of the primary IOL (or for example, lens 154) is 20 D. The approximate angular magnification is 3× at a distance of 50 cm compared to an equivalent eye with a 20 D IOL.

3×/20 D Intraocular Telescope - 50 cm reading distance

| | | | | Diam | Thickness |
|---|---|---|---|---|---|
| Surface | Radius(mm) | Conic K | Material | (mm) | (mm) |
| 152, 206 | 1.5 | −1.659937 | PMMA | 1.5 | 0.6 |
| Anterior | | | | | |
| 152, 206 | −0.75 | −1.659937 | 1.336 | 1.5 | 2.0 |
| Posterior | | | | | |
| 156, 204 | −0.707385 | −5.180637 | PMMA | 1.0 | 0.3 |
| Anterior | | | | | |
| 156, 204 | 0.707385 | −5.180637 | 1.336 | 1.0 | 0.5 |
| Posterior | | | | | |
| 154, 202 cent | −0.530481 | 0 | PHMA | 1.0 | 0.3 |
| S0 | | | | | |
| 154, 202 | 0.530481 | 0 | 1.336 | 1.0 | |
| cent S1 | | | | | |
| 154, 202 | 12.215 | 0 | PHMA | 6.0 | |
| periph S0 | | | | | |
| 154, 202 | −12.215 | 0 | 1.336 | 6.0 | |
| periph S1 | | | | | |

Note:
K = −e²

3×/20 D Intraocular Telescope (0.5 mm space
between third lens and second-slightly larger
angular magnification) 50 cm reading distance

| | | | | Diam | Thickness |
|---|---|---|---|---|---|
| Surface | Radius(mm) | Conic K | Material | (mm) | (mm) |
| 152, 206 | 1.5 | −1.638534 | PMMA | 1.5 | 0.6 |
| Anterior | | | | | |
| 152, 206 | −0.75 | −1.638534 | 1.336 | 1.5 | 2.0 |
| Posterior | | | | | |
| 156, 204 | −0.604687 | −3.024514 | PMMA | 1.0 | 0.3 |
| Anterior | | | | | |
| 156, 204 | 0.604687 | −3.024514 | 1.336 | 1.0 | 1.0 |
| Posterior | | | | | |
| 154, 202 | −0.596196 | 0 | PHMA | 1.0 | 0.3 |
| cent S0 | | | | | |
| 154, 202 | 0.596196 | 0 | 1.336 | 1.0 | |
| cent S1 | | | | | |
| 154, 202 | 12.215 | 0 | PHMA | 6.0 | |
| periph S0 | | | | | |
| 154, 202 | −12.215 | 0 | 1.336 | 6.0 | |
| periph S1 | | | | | |

Note:
K = −e²

The following table illustrates an example with a 25 cm reading distance.

3×/20 D Intraocular Telescope (0.5 mm space between
the third lens and the second lens - slightly larger
angular magnification) 25 cm reading distance

| | | | | Diam | Thickness |
|---|---|---|---|---|---|
| Surface | Radius(mm) | Conic K | Material | (mm) | (mm) |
| 152, 206 | 1.5 | −1.635769 | PMMA | 1.5 | 0.6 |
| Anterior | | | | | |
| 152, 206 | −0.75 | −1.635769 | 1.336 | 1.5 | 2.0 |
| Posterior | | | | | |
| 156, 204 | −0.619793 | −3.112455 | PMMA | 1.0 | 0.3 |
| Anterior | | | | | |
| 156, 204 | 0.619793 | −3.112455 | 1.336 | 1.0 | 1.0 |
| Posterior | | | | | |
| 154, 202 | −0.601335 | 0 | PHMA | 1.0 | 0.3 |
| cent S0 | | | | | |
| 154, 202 | 0.601335 | 0 | 1.336 | 1.0 | |
| cent S1 | | | | | |
| 154, 202 | 12.215 | 0 | PHMA | 6.0 | |
| periph S0 | | | | | |
| 154 periph S1 | −12.215 | 0 | 1.336 | 6.0 | |

These examples are not meant to limit the scope of the invention and are merely to facilitate understanding of the invention. The intraocular telescope embodiments described herein can have any suitable dimensions, sizes or configurations suitable for correction and/or changing the refractive properties of the eye.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method of altering vision in an eye, comprising the step of
inserting into the eye a supplemental lens in series with an implanted Galilean telescopic intraocular lens, said Galilean telescopic intraocular lens is adapted to form a first image from a first field of vision and a portion of a second lens is adapted to form a second image from a second field of vision;
wherein said supplemental lens facilitates focusing light through the Galilean telescopic intraocular lens to form the first image and renders the second image substantially imperceptible.

2. A method according to claim 1, wherein
said lens is implanted in the cornea.

3. A method according to claim 1, wherein
said lens is implanted in the anterior chamber.

4. A method according to claim 1, wherein
said lens is implanted in the posterior chamber.

5. A method according to claim 1, wherein
said second lens is an artificial lens and said supplemental lens is coupled to said artificial lens.

6. A method according to claim 1, wherein
said lens is coupled to said Galilean telescopic intraocular lens.

7. A method according to claim 1, wherein
said lens is a concave lens.

8. A method according to claim 1, wherein
said lens is inserted such that natural fluid from the eye can pass between said supplemental lens and said Galilean telescopic intraocular lens.

9. An implantable lens for converting a lens system from a multifocal lens system to a unifocal lens system, said multifocal lens system including a Galilean telescopic intraocular lens configured to form a first image from a first field of vision and a portion of a second lens configured to form a second image from a second field of vision, the implantable lens comprising:
a plus lens adapted to be implanted in series with the Galilean telescopic intraocular lens such that said implantable lens facilitates focusing light through the Galilean telescopic intraocular lens to form a first image and renders the second image substantially imperceptible.

10. An implantable lens according to claim 9, wherein
said plus lens is implanted in the cornea.

11. An implantable lens according to claim 9, wherein
said second lens is an artificial lens and said plus lens is coupled to said artificial lens.

12. An implantable lens according to claim 9, wherein
said plus lens is coupled to said Galilean telescopic intraocular lens.

13. An implantable lens according to claim 9, wherein
said plus lens is inserted such that natural fluid from the eye can pass between said plus lens and said Galilean telescopic intraocular lens.

14. A method of converting a lens system from a multifocal lens system to a unifocal lens system, said multifocal lens system including a Galilean telescopic intraocular lens configured to form a first image from a first field of vision and a portion of a second lens configured to form a second image from a second field of vision, the method comprising the step of
implanting into the eye a supplemental lens in series with the Galilean telescopic intraocular lens such that the lens facilitates focusing light through the Galilean telescopic intraocular lens to form the first image and renders the second image substantially imperceptible.

15. A method according to claim 14, wherein
said supplemental lens is implanted in the cornea.

16. A method according to claim 14, wherein
said second lens is an artificial lens and said supplemental lens is coupled to said artificial lens.

17. A method according to claim 14, wherein
said supplemental lens is coupled to said Galilean telescopic intraocular lens.

18. A method according to claim 14, wherein
said supplemental lens is a convex lens.

19. A method according to claim 14, wherein
said supplemental lens is inserted such that natural fluid from the eye can pass between said supplemental lens and said Galilean telescopic intraocular lens.
